(19) United States
(12) Patent Application Publication
(10) Pub. No.: US 2026/0007307 A1
(43) Pub. Date: Jan. 8, 2026

(54) ADJUSTMENT MODULE FOR MEDICAL INSTRUMENT AND MEDICAL INSTRUMENT

(71) Applicant: Micro-Tech (Nanjing) Co., Ltd., Nanjing (CN)

(72) Inventors: Qiang Wang, Nanjing (CN); Zhi Tang, Nanjing (CN); Yunfeng Zhou, Nanjing (CN); Mingqiao Fan, Nanjing (CN); Huan Xie, Nanjing (CN)

(21) Appl. No.: 19/196,226

(22) Filed: May 1, 2025

(30) Foreign Application Priority Data
Jul. 4, 2024 (CN) 2024215708888

(51) Int. Cl. A61B 1/045; A61B 1/00

(52) U.S. Cl. CPC A61B 1/045; A61B 1/00066; A61B 1/00147

(57) ABSTRACT

Provided are adjustment module for medical instrument, and medical instrument. The adjustment module includes: operational unit, provided on housing of the medical instrument, where the operational unit can move by first distance under external force; and transmission unit, connected to the operational unit, where the transmission unit includes driving member, fixing member and driven member, the driving member is in transmission connection with the fixing member and the driven member, respectively, the driven member is connected to the working module, the fixing member is fixedly provided on the housing, where the driving member is in transmission connection with the operational unit and is driven by the operational unit to move by second distance along the fixing member, and the driven member can be driven by the driving member to move by third distance that is greater than the first distance and the second distance.

### CROSS-REFERENCE TO RELATED APPLICATIONS

The present disclosure claims the priority to the Chinese patent application with the filling No. 2024215708888, filed on Jul. 4, 2024, the entire content of which is incorporated herein by reference for all purposes.

### TECHNICAL FIELD

The present disclosure relates to the technical field of medical instruments, and particularly to an adjustment module for a medical instrument and a medical instrument.

### BACKGROUND ART

An endoscope is medical instrument that integrates conventional optics, ergonomics, precision machinery, modern electronics, mathematics, software and so on. The endoscope is equipped with an image sensor, an optical lens, a light source for illumination, a mechanical device, etc., and it enters the body through a natural luminal channel of a living body to perform corresponding operations. As a medical instrument with extremely high precision, the endoscope can be used for resection of tissues, such as polyps or other suspected lesions, or the endoscope is used for observing relevant tissue. During tissue resection, a cauterization catheter equipped with a snare is inserted into the endoscope, or the snare is directly extended into the living body so as to remove and cauterize tissue. In many surgical situations, medical care personnel preferentially use a tool executing multiple tasks with minimal motion, and a range of motion of each medical care personnel when operating the endoscope is different due to different habits and different hand sizes of the medical care personnel.

In the related art, a working end occasionally requires a considerable movement distance, while the operating end of the endoscope cannot be suitable for most medical care personnel when an action range is too large, and it is challenging to manipulate single-handedly, which makes it hard to sense a sufficient movement distance of a distal end of the endoscope during operation, thus compromising precision in distal operation, and in severe cases, severance of polyps or tissues may occur before cautery current application, leading to hemorrhaging and other harmful consequences.

### SUMMARY

Based on this, it is necessary to provide an adjustment module for a medical instrument and a medical instrument, with convenient operation and wide applicability to solve the above problems.

In one aspect, an adjustment module for a medical instrument is provided, including:
an operational unit, provided on a housing of the medical instrument, where the operational unit can move by a first distance under an external force; and
a transmission unit, connected to the operational unit, where the transmission unit includes a driving member, a fixing member and a driven member, the driving member is in transmission connection with the fixing member and the driven member, respectively, the driven member is connected to the working module, the fixing member is fixedly provided on the housing, where the driving member is in transmission connection with the operational unit and is driven by the operational unit to move by a second distance along the fixing member, and the driven member can be driven by the driving member to move by a third distance that is greater than the first distance and the second distance.

In one of the embodiments, the driven member and the fixing member are racks with different lengths, the driven member and the fixing member are parallel and spaced apart from each other, and the driving member is a gear engaged with the racks respectively; a total length of the fixing member limits an upper limit of the second distance, and a total length of the driven member limits an upper limit of the third distance.

In one of the embodiments, the third distance is at least twice the second distance.

In one of the embodiments, the adjustment module further includes a support unit and a shaft provided at a centerline of the driving member, one end of the support unit is connected to the operational unit, and the other end is connected to the shaft of the driving member, the operational unit can slide by the first distance in a first direction along the housing, and the driving member rotates around the shaft and moves by the second distance on the fixing member in the first direction, the first distance being equal to the second distance.

In one of the embodiments, the support unit includes a first support and a second support, the first support is sleeved outside the driving member, two ends of the shaft are connected to the first support, and the second support is connected to the first support and the operational unit.

In one of the embodiments, the fixing member and the housing are integrally molded.

In one of the embodiments, the operational unit includes an operational part and an electric plug, the operational part forms an accommodating cavity communicating with the housing, the electric plug is provided in the accommodating cavity, and the electric plug can move with the operational part and be electrically connected to the transmission unit.

In one of the embodiments, the fixing member is configured to fix one end of the driving member, the driving member and the driven member can synchronously rotate around the fixing member, and the driven member is provided away from the fixing member relative to the driving member; and the driven member is connected to the working module, and the driven member can be driven by the driving member to move by the third distance.

In another aspect, a medical instrument is provided, using the above adjustment module.

In one of the embodiments, the medical instrument is a snare, the medical instrument further includes a housing and a working module, where the working module includes a cable and a ring provided on the housing, a proximal end of the cable is connected to the driven member, thereby enabling the cable to move with the driven member relative to the housing, and a distal end of the cable is connected to the ring.

With the above adjustment module, when the operational unit is moved by the first distance in the first direction relative to the operational handle, due to the structure of the transmission unit, the driven member connected to the working module can be driven by the driving member to move by the third distance that is greater than the first distance and the second distance, thus enabling a longer movement distance of the working module with a smaller operational amplitude, saving operation actions of operators, and being suitable to a wide range of various types of operators.

### BRIEF DESCRIPTION OF DRAWINGS

FIG. 1 is a structural schematic diagram of a snare in an embodiment of the present disclosure.

FIG. 2 is a cross-sectional structural schematic diagram of an adjustment module for a snare in an embodiment of the present disclosure.

FIG. 3 is an enlarged view of part A in FIG. 2.

FIG. 4 is a structural schematic diagram of a driving member and a support unit in an embodiment of the present disclosure.

### DETAILED DESCRIPTION OF EMBODIMENTS

In order to make the above objectives, features and advantages of the present disclosure more apparent and understandable, embodiments of the present disclosure are described in detail below in combination the drawings. Many specific details are set forth in the following description in order to provide a thorough understanding of the present disclosure. However, the present disclosure can be implemented in many other modes different from those described herein. Those skilled in the art could make similar improvements without departing from the connotation of the present disclosure. Therefore, the present disclosure is not limited to the embodiments disclosed below.

In the description of the present disclosure, it should be understood that orientation or positional relations indicated by the terms “center”, “longitudinal”, “lateral”, “length”, “width”, “thickness”, “upper”, “lower”, “front”, “back”, “left”, “right”, “vertical”, “horizontal”, “top”, “bottom”, “inner”, “outer”, “clockwise”, “anticlockwise”, “axial”, “radial”, and “circumferential” and so on, if appear, are based on orientation or positional relations as shown in the drawings, merely for facilitating the description of the present disclosure and simplifying the description, rather than indicating or implying that related devices or elements have to be in the specific orientation or configured and operated in a specific orientation, and thus they should not be construed as limitation to the present disclosure.

Besides, the terms “first” and “second”, if appear, are merely used for descriptive purpose, but should not be construed as indicating or implying importance in the relativity or implicitly indicating the number of a related technical feature. Therefore, a feature defined with the term “first” or “second” may explicitly or implicitly mean that at least one such feature is included. In the description of the present disclosure, if the term “multiple (a plurality of)” appears, it means at least two, for example, two and three, unless otherwise defined explicitly.

In the present disclosure, unless otherwise specified and defined explicitly, the terms “mount”, “join”, “connect”, etc. should be construed in a broad sense. For example, it may be a fixed connection, or a detachable connection, or an integrated connection; it may be a mechanical connection, or an electrical connection; it may be a direct connection, or an indirect connection via an intermediary, or inner communication between two elements or interactive relationship between two elements, unless otherwise expressly defined. For those ordinarily skilled in the art, specific meanings of the above-mentioned terms in the present disclosure could be understood according to specific circumstances.

In the present disclosure, unless otherwise specified and defined explicitly, if there appears a description that a first feature is “above” or “below” a second feature or similar description, it may mean that the first feature is in direct contact with the second feature or that the first feature is in contact with the second feature through an intermediary. Moreover, a first feature being “on”, “over” and “above” a second feature may include a case where the first feature is directly or not directly above the second feature, or merely indicates that the first feature is at a horizontal height larger than that of the second feature. The first feature being “under”, “beneath” and “below” a second feature may include a case where the first feature is directly or not directly below the second feature, or merely indicates that the first feature is at a horizontal height smaller than that of the second feature.

It should be noted that, when an element is referred to as “being fixed to” or “being arranged on” another element, the element may be directly on the another element or an intermediate element may also exist. If an element is considered “connected to” another element, it may be directly connected to the another element or there may be an intermediate element therebetween. The terms “vertical”, “horizontal”, “upper”, “lower”, “left”, “right” and similar expressions used herein are for illustrative purposes only and do not mean the only implementation.

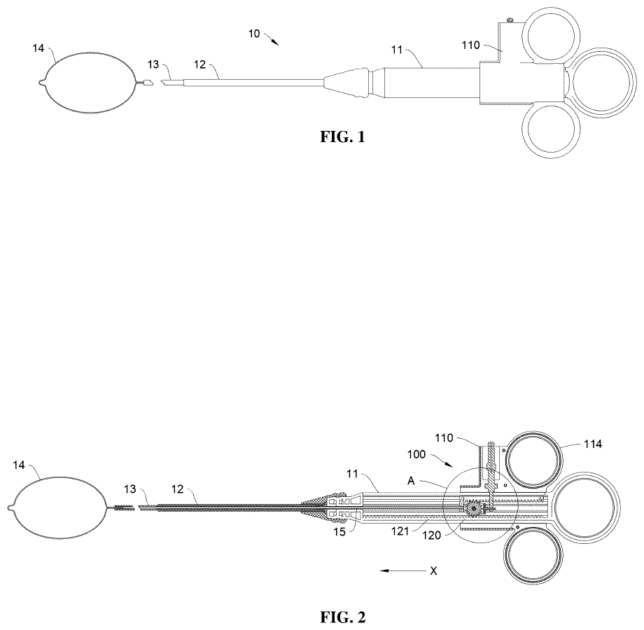

Referring to FIG. 1 to FIG. 4, FIG. 2 shows a structural schematic diagram of an adjustment module 100 for a medical instrument in an embodiment of the present disclosure. The medical instrument provided by an embodiment of the present disclosure can be used together with an endoscope, can also be used separately, and particularly may be a snare 10 for resection of part of tissues, as shown in FIG. 1. The present disclosure is described by taking the medical instrument being the snare 10 as an example, and the adjustment module 100 is applied to the snare 10.

As shown in FIG. 1 and FIG. 2, the snare 10 includes a housing and a working module, where the housing includes an operational handle 11, an insertion portion 12 and a catheter 13, and the working module includes a cable 15 and a ring 14 provided on the housing. The catheter 13 is made from a flexible insulator in an elongated tubular shape, and the catheter 13 can be inserted into an instrument insertion passage in the insertion portion 12. The working module is provided in the catheter 13, and the working module is partially extended out from a distal end of the catheter 13. The catheter 13 is equipped with the operational handle 11 on a proximal end. The cable 15 and the ring 14 can be electrically conductive. The ring 14 is annular, and the shape of the annular ring 14 can be changed so as to be opened or closed. The cable 15 and the ring 14 are fixed through an electrically conductive connector.

The adjustment module 100 is provided on the housing, mostly at the operational handle 11. Some structures of the adjustment module 100 are slidably provided on the operational handle 11 of the housing. The adjustment module 100 is connected to the working module. When some structures of the adjustment module 100 slide relative to the housing, the working module moves axially in the interior of the catheter 13 (the interior of lumen).

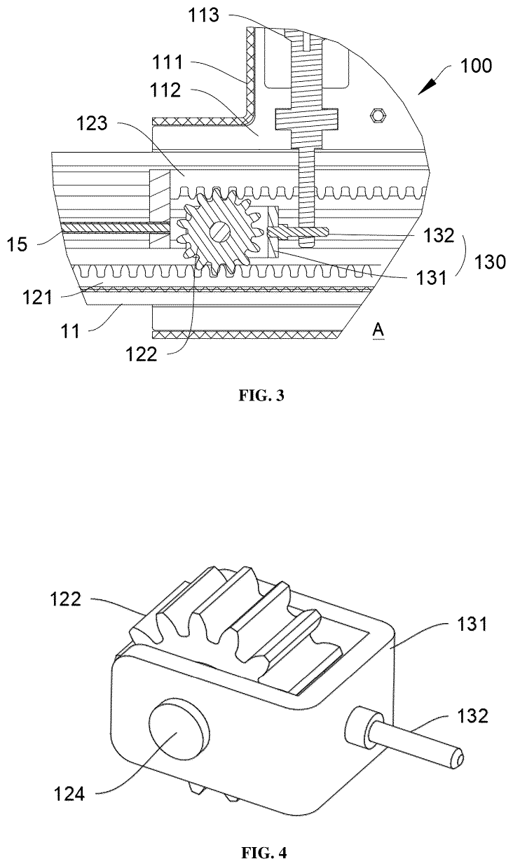

Referring to FIG. 3, FIG. 3 is a partially enlarged structural diagram of part A in FIG. 2. The adjustment module 100 includes an operational unit 110 and a transmission unit 120. The operational unit 110 is provided on the housing of the medical instrument, and the operational unit 110 can move by a first distance under an external force. The transmission unit 120 is connected to the operational unit 110. The transmission unit 120 includes a driving member 122, a fixing member 121 and a driven member 123. The driving member 122 is in transmission connection with the fixing member 121 and the driven member 123, respectively. The driven member 123 is connected to the working module. The fixing member 121 is fixedly provided on the housing. Herein, the driving member 122 is in transmission connection with the operational unit 110 and is driven by the operational unit 110 to move by a second distance along the fixing member 121. The driven member 123 can be driven by the driving member 122 to move by a third distance that is greater than both the first distance and the second distance.

The operational unit 110 is provided at the operational handle 11 and has a part that can be operated by an external user or a tool, and the operated operational unit 110 slides by the first distance along the housing. The transmission unit 120 is provided in the operational handle 11 and connects the operational unit 110 and a proximal end of the cable 15. The ring 14 is provided at a distal end of the cable 15. When the cable 15 is driven by the transmission unit 120, the ring 14 can move along an axial direction of the catheter 13.

When using the snare 10, a high-frequency generator is connected, and the catheter 13 is inserted into the instrument insertion passage in the insertion portion 12. A distal end of the insertion portion 12 of the snare 10 is inserted into a body cavity, and thus the catheter 13 is inserted into the body cavity. A distal end of catheter 13 is extended out from the distal end of insertion portion 12, thereby enabling the ring 14 to be extended out from the distal end of the catheter 13. The operational unit 110 is moved by the first distance along an axial direction (a first direction) relative to the operational handle 11. The cable 15 is moved forward in the first direction relative to the catheter 13. In this way, the ring 14 extended out from a front end of the catheter 13 is automatically opened by an elastic force thereof, forming an annular shape. The ring 14 is adjusted to an orientation in which it is easy to be sleeved on polyp, enabling the ring 14 to be easily and reliably sleeved on the polyp. In this state, the operational unit 110 is moved a certain distance in a direction opposite to the first direction relative to the operational handle 11, and then a diameter of the ring 14 is gradually reduced, thus tightening the polyp.

In the above process, when the operational unit 110 is moved by the first distance in the first direction relative to the operational handle 11, due to the structure of the transmission unit 120, the driven member 123 connected to the working module can be driven by the driving member 122 to move by the third distance that is greater than the first distance and the second distance, thus enabling a longer movement distance of the working module with a smaller operational amplitude, saving operation actions of operators, and being suitable to a wide range of various types of operators.

In one of the embodiments, the driven member 123 and the fixing member 121 are racks with different lengths, the driven member 123 and the fixing member 121 are parallel and spaced apart from each other, and the driving member 122 is a gear engaged with the racks respectively. A total length of the fixing member 121 limits an upper limit of the second distance, and a total length of the driven member 123 limits an upper limit of the third distance. Further, the third distance is at least twice the second distance.

Specifically, the driven member 123 and the fixing member 121 are provided in parallel at an interval in the first direction inside the operational handle 11 of the housing, teeth of the driven member 123 and the fixing member 121 are provided facing each other, and the driving member 122 is engaged with the teeth of the driven member 123 and the fixing member 121 facing each other. When the operational unit 110 is driven to move, the driving member 122 is driven to rotate along the teeth of the fixing member 121, the driving member 122 drives the driven member 123 engaged on the other side to move by the third distance, thus amplifying the stroke by a factor of at least 2. When the gear is rotated counterclockwise in the embodiment shown in the drawing, the driven member 123 is moved by the third distance in the first direction relative to the operational handle 11; when the gear is rotated clockwise in the embodiment shown in the drawing, the driven member 123 is moved by the third distance in a direction opposite to the first direction relative to the operational handle 11.

In one of the embodiments, the fixing member 121 and the housing are integrally molded. In other embodiments, the fixing member 121 and the housing are independent structures, and the fixing member 121 is fixedly provided inside the housing.

In one of the embodiments, the adjustment module 100 further includes a support unit 130 and a shaft 124 provided at a centerline of the driving member 122. One end of the support unit 130 is connected to the operational unit 110, and the other end is connected to the shaft 124 of the driving member 122. The operational unit 110 can slide by the first distance in the first direction along the housing, and the driving member 122 rotates around the shaft 124 and moves by the second distance on the fixing member 121 in the first direction.

Further, the support unit 130 includes a first support 131 and a second support 132. The first support 131 is sleeved outside the driving member 122 and is not in contact with the driving member 122, so as to avoid hindering the rotation of the driving member 122. Two ends of the shaft 124 are connected to the first support 131, and the second support 132 is connected to the first support 131 and the operational unit 110.

As shown in FIG. 4, specifically, the shaft 124 is provided in a direction perpendicular to the first direction, and two ends of the shaft 124 protrude out from the driving member 122 to be connected to the support unit 130. The first support 131 is in an arched shape, and the first support 131 includes two first parts provided on the two end surfaces of the driving member 122 and a second part connecting the first parts, and the second part correspondingly encloses a peripheral surface of the driving member 122. An end of each of the two first parts is connected to two ends of the shaft 124, and the other end of each of the two first parts is connected to two ends of the second part. The second support 132 is rod-shaped, and the second support 132 is provided in the middle of the second part in the first direction.

In some embodiments, the operational unit 110 includes an operational part 111 and an electric plug 113, the operational part 111 forms an accommodating cavity 112 communicating with the housing, the electric plug 113 is provided in the accommodating cavity 112, and the electric plug 113 can move with the operational part 111 and be electrically connected to the transmission unit 120, thereby being electrically connected to the working module. The electric plug 113 is configured to connect an external high-frequency generator, and the high-frequency generator supplies power to the snare 10 for resection of corresponding tissue. In this state, a high-frequency current moves and flows from the high-frequency generator to the electric plug 113, and flows from the electric plug 113 to the ring 14 via the transmission unit 120 and the cable 15. Finally, the polyp is severed, and the supply of the high-frequency current is stopped after severance of the polyp.

The electric plug 113 is provided in a direction perpendicular to the first direction. The electric plug 113 extends from an opening of the operational part 111 into the accommodating cavity 112 and the operational handle 11, and passes through the operational handle 11 to be connected to the second support 132 of the support unit 130. The driven member 123 or the fixing member 121 is provided with a clearance from the electric plug 113.

The operational part 111 is in a cylindrical shape, and the operational part 111 is directly sleeved on an outer surface of the operational handle 11. The operational unit 110 further includes an annular portion parallel to the operational part 111. The annular portion is configured to place index finger, middle finger, etc., so as to facilitate the user's operation.

In other embodiments, the snare 10 is not provided with the electric plug 113, and the operational part 111 is provided therein with a structure for connecting the support unit 130.

In one of the embodiments, not shown in the drawings, the fixing member is configured to fix one end of the driving member, the driving member and the driven member can synchronously rotate around the fixing member, and the driven member is provided away from the fixing member relative to the driving member. The driven member is connected to the working module, and the driven member can be driven by the driving member to move by the third distance.

Specifically, the fixing member acts as a rotation center of the driving member, the transmission unit further includes a swing arm, and the swing arm connects the fixing member, the driven member, and the driving member together, thus enabling the driven member and the driving member to rotate synchronously around the fixing member as a center of circle. The driving member and the driven member are fixed in a track provided in the first direction, an external force directly drives the driving member, and through the swing arm and the fixing member, the swing arm swings around the fixing member, and drives the driven member to move by the third distance in the first direction. As the driven member is farther away from the center of circle (the fixing member) than the driving member, when the driving member moves by the second distance, the third distance of movement of the driven member at a further outer ring is greater than the second distance, thus realizing a result of enlarging the stroke.

Various technical features of the above embodiments can be combined at will. In order to make the description concise, not all possible combinations of the technical features in the above embodiments are described. However, the combinations of these technical features should be considered as falling within the scope of the present description, as long as there is no contradiction.

Only several embodiments of the present disclosure are illustrated in the above, the description of which is specific and detailed, but should not be construed as limiting the scope of the present disclosure. It should be noted that various variations and improvements could be made by those skilled in the art without departing from the concept of the present disclosure, all of which fall within the scope of protection of the present disclosure. Therefore, the scope of protection of the present disclosure shall be determined by the appended claims.

1. An adjustment module for a medical instrument, comprising:
an operational unit, provided on a housing of the medical instrument, wherein the operational unit can move by a first distance under an external force; and
a transmission unit, connected to the operational unit, wherein the transmission unit comprises a driving member, a fixing member and a driven member, the driving member is in transmission connection with the fixing member and the driven member, respectively, the driven member is connected to a working module, and the fixing member is fixedly provided on the housing, wherein the driving member is in transmission connection with the operational unit and is driven by the operational unit to move by a second distance along the fixing member, and the driven member can be driven by the driving member to move by a third distance, wherein the third distance is greater than both the first distance and the second distance.

2. The adjustment module for a medical instrument according to claim 1, wherein the driven member and the fixing member are racks with different lengths, the driven member and the fixing member are parallel and spaced apart from each other, and the driving member is a gear engaged with the racks respectively; and a total length of the fixing member limits an upper limit of the second distance, and a total length of the driven member limits an upper limit of the third distance.

3. The adjustment module for a medical instrument according to claim 2, wherein the third distance is at least twice the second distance.

4. The adjustment module for a medical instrument according to claim 2, wherein the adjustment module further comprises a support unit and a shaft provided at a centerline of the driving member, one end of the support unit is connected to the operational unit, and the other end is connected to the shaft of the driving member; the operational unit can slide by the first distance in a first direction along the housing; and the driving member rotates around the shaft and moves by the second distance on the fixing member in the first direction.

5. The adjustment module for a medical instrument according to claim 4, wherein the support unit comprises a first support and a second support, the first support is sleeved outside the driving member, two ends of the shaft are connected to the first support, and the second support is connected to the first support and the operational unit.

6. The adjustment module for a medical instrument according to claim 2, wherein the fixing member and the housing are integrally molded.

7. The adjustment module for a medical instrument according to claim 1, wherein the operational unit comprises an operational part and an electric plug, the operational part forms an accommodating cavity communicating with the housing, the electric plug is provided in the accommodating cavity, and the electric plug can move with the operational part and be electrically connected to the transmission unit.

8. The adjustment module for a medical instrument according to claim 1, wherein the fixing member is configured to fix one end of the driving member, the driving member and the driven member can synchronously rotate around the fixing member, and the driven member is provided away from the fixing member relative to the driving member; and the driven member is connected to the working module, and the driven member can be driven by the driving member to move by the third distance.

9. A medical instrument, using the adjustment module according to claim 1.

10. The medical instrument according to claim 9, wherein the medical instrument is a snare, the medical instrument further comprises the housing and the working module, wherein the working module comprises a cable and a ring provided on the housing, a proximal end of the cable is connected to the driven member, thereby enabling the cable to move with the driven member relative to the housing, and a distal end of the cable is connected to the ring.

11. The medical instrument according to claim 9, wherein the driven member and the fixing member are racks with different lengths, the driven member and the fixing member are parallel and spaced apart from each other, and the driving member is a gear engaged with the racks respectively; and a total length of the fixing member limits an upper limit of the second distance, and a total length of the driven member limits an upper limit of the third distance.

12. The medical instrument according to claim 11, wherein the third distance is at least twice the second distance.

13. The medical instrument according to claim 11, wherein the adjustment module further comprises a support unit and a shaft provided at a centerline of the driving member, one end of the support unit is connected to the operational unit, and the other end is connected to the shaft of the driving member; the operational unit can slide by the first distance in a first direction along the housing; and the driving member rotates around the shaft and moves by the second distance on the fixing member in the first direction.

14. The medical instrument according to claim 13, wherein the support unit comprises a first support and a second support, the first support is sleeved outside the driving member, two ends of the shaft are connected to the first support, and the second support is connected to the first support and the operational unit.

15. The medical instrument according to claim 11, wherein the fixing member and the housing are integrally molded.

16. The medical instrument according to claim 9, wherein the operational unit comprises an operational part and an electric plug, the operational part forms an accommodating cavity communicating with the housing, the electric plug is provided in the accommodating cavity, and the electric plug can move with the operational part and be electrically connected to the transmission unit.

17. The medical instrument according to claim 9, wherein the fixing member is configured to fix one end of the driving member, the driving member and the driven member can synchronously rotate around the fixing member, and the driven member is provided away from the fixing member relative to the driving member; and the driven member is connected to the working module, and the driven member can be driven by the driving member to move by the third distance.
